(19) United States
(12) Patent Application Publication
(10) Pub. No.: US 2018/0289311 A1
(43) Pub. Date: Oct. 11, 2018

(54) EEG RECORDING DEVICE

(71) Applicant: EPIC Neuro, Inc., Fountain Valley, CA (US)

(72) Inventor: James William Phillips, Fountain Valley, CA (US)

(73) Assignee: EPIC Neuro, Inc., Fountain Valley, CA (US)

(21) Appl. No.: 15/945,511

(22) Filed: Apr. 4, 2018

Related U.S. Application Data
(60) Provisional application No. 62/482,579, filed on Apr. 6, 2017.

(51) Int. Cl. A61B 5/00; A61B 5/04; A61B 5/07; A61B 90/10; A61B 34/10

(52) U.S. Cl. CPC A61B 5/4094; A61B 5/04001; A61B 5/076; A61B 2090/103; A61B 5/7282; A61B 5/7405; A61B 34/10; A61B 90/10

(57) ABSTRACT

A device is described, which is fully implantable and records the EEG of a person, where the device comprises a subcranial probe containing one or more electrodes, and an extracranial portion, which includes at least one electrode. The subcranial probe proceeds through an opening in the skull. The intracranial portion comprises a seal, which prevents fluid ingress or egress between the subcranial portion and the extracranial portion. Due to the high electrical resistance of the skull, the seal effectively isolates the extracranial electrode from the intracranial electrodes, making the extracranial electrode an effective ground reference for EEG recording.

### CROSS-REFERENCE TO RELATED APPLICATIONS

This application claims the benefit of Provisional No. 62/482,579 (Attorney Docket No. 51576-704.101), filed Apr. 6, 2017, the entire content of which is incorporated herein by reference.

### BACKGROUND OF THE INVENTION

EEG recording is traditionally used in diagnosing and localizing seizure activity associated with epilepsy. It has been shown in clinical studies that seizure activity can be significantly reduced when the brain tissue in the region of the seizure focus is either stimulated through neuromodulation, or resected by removing a portion of brain tissue in the region. In order to accurately localize seizure activity, invasive EEG recording is often necessary. Such recording can be performed using an Electrocorgicography (ECoG) strip or grid of electrodes placed on the cortical surface to measure cortical EEG activity, or using depth electrodes to record EEG activity from deeper structures, such as the thalamus or anterior cingulate.

Stereotactic EEG (SEEG) is a procedure whereby a depth probe is inserted through a burr-hole into a person's brain in order to record EEG from a target location. The depth probe may have one or more electrodes spaced along the length of the probe, which allows EEG to be recorded between any two of the electrodes. A cable from the depth probe proceeds percutaneously through a small incision in the scalp, and is attached to an EEG amplifier. The EEG amplifier is used in conjunction with an EEG recorder, in order to create long-term recordings of the person's EEG. Often, up to 14 depth probes are inserted into the patient's brain at specific locations, allowing for a sparse mapping of brain activity in cortical and subcortical regions.

The major downside to the ECoG or SEEG devices is that a lead protrudes through the patient's scalp. This requires the patient to stay within a short distance from an external EEG recorder. A chance of infection or pulling on the cable is also a risk to the patient. In general, a patient must sit in a hospital Epilepsy Monitoring Unit (EMU) or intensive care unit (ICU), where EEG is continuously monitored. For patients with epilepsy, they must wait for a seizure to occur to localize the seizure focus. Patients often titrate or stop their anti-epilepsy medication in order to bring about a seizure more quickly. When this is done, the seizure is often severe, which can result in status epilepticus, an added risk to the patient.

A fully implantable EEG recording system would obviate the need for the patient to remain at the hospital to await a seizure. Instead, the patient would be allowed to return home, resuming normal daily activities while awaiting a seizure.

Implantable medical devices have been proposed that allow for long term fully implantable EEG recording. In one example, the Responsive Neuro Stimulation (RNS) device, offered by NeuroPace, Inc. (Sunnyvale, Calif.) incorporates an EEG recording system as part of the neuromodulator, in order to detect when seizures occur using an algorithm. An implantable EEG Seizure Advisory System (SAS) designed for seizure prediction was designed by NeuroVista, Inc. (Seattle, Wash.). Both systems use cortical strip electrodes or depth electrodes to make the recording. The RNS device allows for recording between any two electrodes, using a third more remote electrode as the ground. The implantable SAS used a remote unit located in the pectoral region with a lead that led to a strip electrode placed on the cortical surface. An electrode on the remote unit served as a ground.

### SUMMARY OF THE INVENTION

In broad terms, the present invention is a device, which is fully implantable in a person, and records the EEG of a person, and comprises an energy storage module, control logic, memory, an EEG amplifier, a subcranial electrode, an extracranial electrode, and an intercranial portion, the intercranial portion passing through an opening in the skull and comprising a seal, wherein the seal prevents electric current from passing around the intercranial portion through the opening in the skull.

The EEG recording may be performed by sensing electric potential between any two electrodes, potentially with a third electrode acting as a ground reference. In one aspect, the EEG is recorded between two subcranial electrodes, with the extracranial electrode acting as a ground reference. In another aspect, the EEG is recorded between the subcranial electrode and the extracranial electrode.

In order to record EEG and provide other functionality, it is critical that the device have an energy storage module. In one aspect, the energy storage module is a battery. In another aspect, the energy storage module is a capacitor.

The device of the present invention may incorporate logic to allow a marker to be included with the EEG recording, which signifies a significant event. For example, a person may wish to include an EEG marker when a seizure is imminent, or when a seizure has just finished. In one aspect of the device, the control logic comprises a means whereby the person can record a marker on the EEG recording. In this case, a means is required to allow the person to specify when a marker should be included. In one aspect of the device, the means of recording an EEG marker is initiated by placing a permanent magnet close to the device.

It is necessary to transfer EEG recording data from the device to an external module, such as a computer. This transfer can occur at regular intervals while the EEG is being recorded, at the end of an EEG recording period, or it can occur after the device has been removed from the person. In one aspect of the device, the control logic comprises a means of sending the recorded EEG data to an external device. Such a transfer can be done wirelessly while still implanted. It could also be done wirelessly or wired after the device has been removed from the person. In one aspect of the device, the means of sending the recorded EEG is through wireless telemetry. In another aspect of the device, the means of sending the recorded EEG is through a wired connection.

A single device may be implanted, or multiple devices may be implanted in a person, in order to get higher resolution EEG mapping of brain activity. If multiple devices are implanted, then the extracranial electrodes could be connected together in a wired system, in order to reduce the impedance between them. In one aspect of the device, the extracranial electrode is electrically connected to the extracranial electrode of a nearby device.

It may be necessary to inform the person, caregiver, or clinical staff the status of the device, or the occurrence of a specific event. In one aspect of the device, the device comprises a speaker to communicate events to a person. In one aspect of the device, the speaker is used to indicate recording start, recording stop, battery level, or EEG marker set.

The device may be implanted using a stereotactic procedure, in order to ensure proper placement of the subcranial probe. It may be necessary for an anchor shaft to be fixed to the device, to give the surgeon a mechanism to guide the device to the correct position. In one aspect of the device, the device comprises a removable anchor shaft that allows for stereotactic implantation of the device.

The EEG recording mechanism will contain an EEG amplifier, control logic, memory, and an energy storage module, as well as an extracranial electrode. This may be enclosed in a case and be situated outside the skull and beneath the scalp of the person. In one aspect of the device, the energy storage module, control logic, memory, EEG amplifier, and extracranial electrode are contained in an enclosure that is located in the extracranial region under the scalp. Since the optimal location for the subcranial probe may not be perpendicular to the skull, it may be necessary for the case to tilt to allow it to lie flat against the skull even if the subcranial probe is not perpendicular to the skull. In one aspect of the device, the container is configured to tilt, to allow the intercranial portion to pass through the skull at an angle that is not perpendicular to the skull.

The subcranial probe may be of a certain length and comprise a certain number of electrodes, spaced along the probe length. In one aspect of the device, the subcranial electrode is part of a probe, which proceeds a distance into the brain. If the probe is short, it may not penetrate the dura. In one aspect of the device, the probe does not penetrate the dura. In one aspect of the device, the probe penetrates the dura.

The device may be used to record EEG of the person for a period of time. One application of the device is for a person who has seizures to record the EEG in order to localize seizure focus. In this case, several devices may be implanted in order to get an EEG mapping of the brain. In one aspect of the device, the device is used to localize seizure focus.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is a drawing of one aspect of the device, in which two devices are implanted in the brain of a person, showing the seal surrounding the intracranial portion in the opening of the skull.

FIG. 2 is a drawing of one aspect of the device, in which the side and top views are shown of the device.

FIG. 3 is a drawing of one aspect of the device, in which an anchor shaft is included as part of one of the devices in order to allow stereotactic implantation of the device.

FIG. 4 is a block diagram of one aspect of the device, in which electrodes are connected to an EEG amplifier, which communicates with EEG recording logic and memory storage, as well as telemetry and supporting components.

FIG. 5 is a drawing of one aspect of the device, in which two devices are implanted in the brain of a person, where the extracranial electrodes are connected by a wire, in order to reduce the impedance between the two electrodes.

### DETAILED DESCRIPTION OF THE INVENTION

While certain embodiments have been provided and described herein, it will be readily apparent to those skilled in the art that such embodiments are provided by way of example only. It should be understood that various alternatives to the embodiments described herein may be employed, and are part of the invention described herein.

Provided herein is a device whereby the EEG of a person is recorded using one or more subcranial electrodes and one or more extracranial electrodes. The subcranial electrodes are electrically connected to the EEG recorder through an intercranial portion of the device, which is located in an opening in the skull, such as a burr-hole or craniotomy. The device can record the EEG signal as the potential difference between any two subcranial electrodes, or between a subcranial electrode and the extracranial electrode. In order to provide the best possible signal, it is necessary that electrical currents generated by brain activity be as electrically isolated as possible from the extracranial electrode. Therefore, the intercranial portion comprises a seal, which fills the space between the intercranial portion and the edge of the opening in the skull, which prevents fluid ingress and egress through the opening in the skull and prevents electric current from flowing through cerebrospinal fluid (CSF) or other fluid which would otherwise fill the space.

In a preferred embodiment of the device, the subcranial electrodes are contained in a probe, which proceeds a distance into the brain. The electrodes may be evenly spaced along the probe, in order to record EEG from several locations in the brain. In one aspect, a single EEG recording is made by sensing the potential difference between two electrodes. In another aspect, multiple EEG recordings are made, by sensing the electric potential between various combinations of electrodes.

The device may be used on its own to record EEG, or the device may be used in concert with other devices in order to provide an EEG mapping of brain activity. Due to the high resistivity of the skull and the seal surrounding the intercranial portion, the extracranial electrode of the device is effectively electrically isolated from the subcranial electrodes. This makes the electrode ideal as a ground reference in order to reduce common mode noise in the system. When multiple devices are used together, all extracranial electrodes are effectively electrically isolated from the subcranial electrodes, and they are all at similar electric potential, due to the low resistivity of the scalp to electric current flow. In one aspect, the extracranial electrodes of each device are connected using one or more wires, thereby bringing them all to the same potential, and removing the effect of the electrode-tissue interface.

In a preferred embodiment of the device, the EEG recording means is performed using a battery, EEG amplifier, a processor, and memory, located in an extracranial portion of the device. In one aspect, the extracranial portion is enclosed in a disk-shaped container that rests on the surface of the skull and is covered by the scalp. In one aspect, the extracranial portion is able to tilt, allowing the burr-hole or craniotomy to be created so that the subcranial electrode probe is at an angle of incidence with the skull that is not 90 degrees. This enables a surgeon to more accurately direct the subcranial electrode to the target location.

In one aspect of the device, the battery is rechargeable, allowing the person or caregiver to recharge the battery using an external power source, such as an external magnetic field generator. In an alternate aspect of the device, the battery is not rechargeable, which means that the time the EEG recording is able to be performed is limited due to the battery life of the device.

It is important for the medical staff to evaluate the EEG recording. In one aspect of the device, the EEG is downloaded by removing the device from the patient and inserting it into a fixture, which uses a direct physical connection to transfer the EEG data from memory. In an alternate aspect of the device, the EEG data is transferred using telemetry to a telemetry module external to the body. For example, a coil may be placed close to the device, and a telemetry link established so that data may be transferred while the device is still implanted in the person. In another example, a Bluetooth or wi-fi connection may be made with the device, allowing transfer of EEG data. Since multiple devices may be implanted at one time, it is important that EEG data be transferred from the desired device, without interference from nearby devices. This may be accomplished by assigning each device a unique identifier, which is used to establish a connection. Devices with identifiers other than the desired device would not transmit EEG data or interfere with the connection with the desired device.

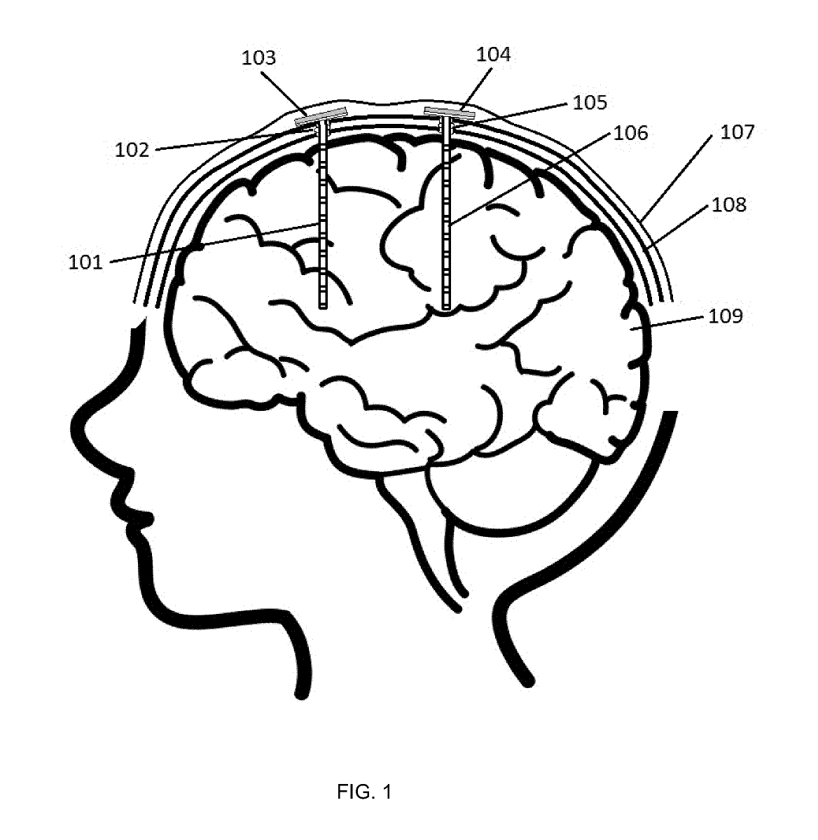

With reference to FIG. 1, a drawing is shown which represents a typical application of the present device. In this, two devices are implanted in a person, each with a subcranial probe (101, 106) inserted through a burr-hole (102, 105) in the skull (108) and proceeding into the brain (109), where each intracranial portion surrounded by a seal, which prevents any fluid ingress or egress through the burr-hole, ensuring that electric current does not flow through the burr-hole. Due to the high electrical resistance of the skull, this makes the extracranial portions effectively electrically isolated from the subcranial probes. Each device includes a disk-shaped container (103, 104), which enclose a battery, EEG amplifier, processor, and memory. The outside of the container includes an extracranial electrode. The disk-shaped extracranial containers are each covered by the person's scalp (107), making them fully implanted. In this application, each subcranial probe comprises multiple subcranial electrodes. An EEG recording may be performed by sensing the voltage potential between any two electrodes, either two subcranial electrodes or between a subcranial electrode and the extracranial electrode. In this application, the disk-shaped containers are tilted so that they lie flat on the outer skull surface. By lying flat, they are able to minimize the bump created in the scalp due to the volume occupied by the disk-shaped containers.

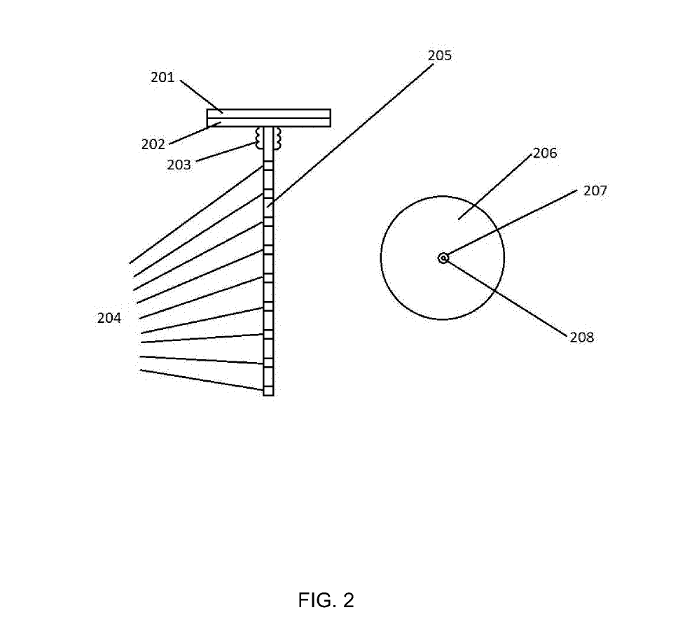

With reference to FIG. 2, a drawing is shown which represents a typical aspect of the present device. In this, a side view (left) and top view (right) are shown. In the side view, a subcranial probe (205) contains 9 electrodes (204) evenly spaced, allowing EEG recording at one or more locations along the probe's length. A battery (201) lies flat at the top of the device and conductive case (202) is beneath. The conductive case acts as an extracranial electrode. The case contains an EEG amplifier, processor, and memory, allowing for EEG recording between any two of the subcranial electrodes with the extracranial electrode acting as a ground reference, or between a subcranial electrode and the extracranial electrode. A seal (203) is included in the intracranial portion to prevent fluid ingress or egress from the burr-hole, thereby providing electrical insulation between the intracranial electrodes and the extracranial electrode. Due to the high resistivity of the skull, the extracranial electrode is effectively isolated from the intracranial electrodes, acting as a proper ground reference.

The top view is shown at the right, showing the top of the battery (206). A hole (207) is contained in the center of the battery, with a threaded hole (208) in the conductive case, allowing for attachment of an anchor shaft, used for stereotactic implantation of the device.

The hole in the battery is wider than the threaded hole in the conductive case, in order to allow the battery and case to tilt a small amount, which allows the conductive case and battery to lie flat against the skull, even if the intracranial probe is not perpendicular to the skull. In order to accomplish the bend, the intracranial portion may include a small flexible portion near the conductive case, allowing the conductive case to tilt.

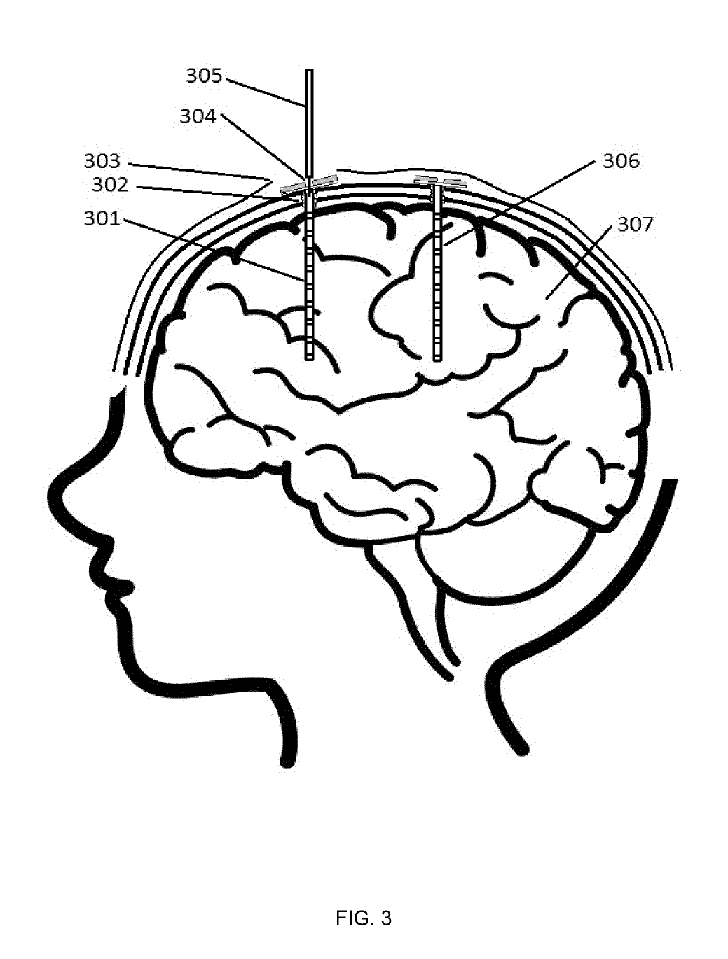

With reference to FIG. 3, a drawing is shown which represents a typical aspect of the present device. In this, two devices are implanted in a person, each with a subcranial probe (301, 306) extending into the brain (307). The left device shows an anchor shaft (305) inserted into a threaded hole (304). The scalp (303) is pulled back to make room for the device to fit. The intracranial portion (302) including a seal is inserted through the burr-hole, and the conductive case and battery are allowed to tilt in order to rest flat against the skull. When the device has been implanted correctly, the anchor shaft may be unscrewed and removed. Including an anchor shaft allows for stereotactic implantation of the device.

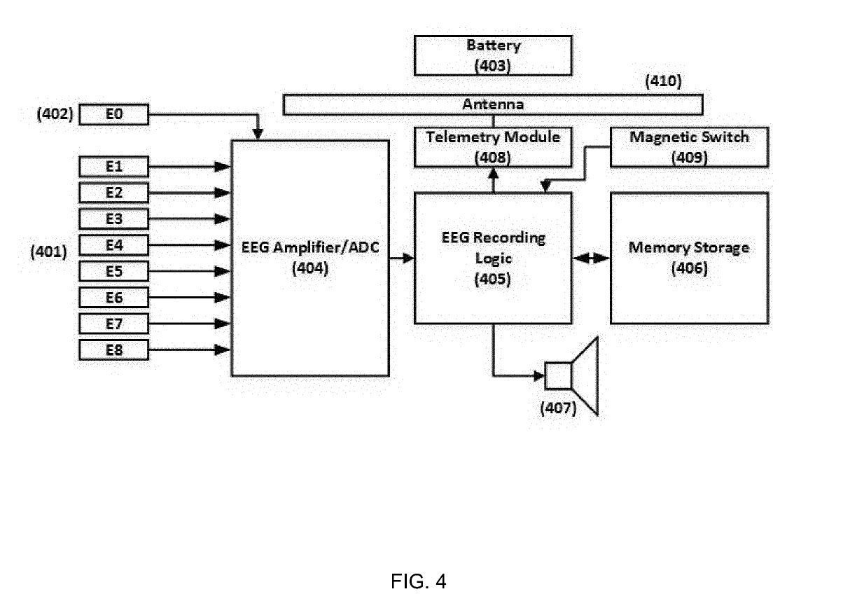

With reference to FIG. 4, a block diagram is shown which represents a typical aspect of the present device. In this, 8 EEG electrodes (401), which are located equally spaced along a subcranial probe. An extracranial electrode (402) is located exterior to the skull and under the scalp, and acts as a ground reference. An EEG Amplifier and analog-to-digital converter (404) amplifies the signal between one or more pairs of electrodes. The EEG recording logic (405) may be a processor, ASIC, FPGA or CPLD, and is responsible for controlling the recording of EEG and storing the EEG in memory storage (406), as well as transmitting the EEG signal outside the person's body using a telemetry module (408). The telemetry module may operate Bluetooth, wi-fi, proprietary protocol, or other protocol to communicate with an external module. The telemetry module transmits data using an antenna (410). The battery (403) supplies power to the device. The magnetic switch (409) may be used by the person, caregiver, or surgeon to perform a variety of functions, including initiating recording, pausing recording, stopping recording, initiating telemetry, or recording a marker indicating the occurrence of an event, such as a seizure. The speaker (407) may be used to signal a variety of information to the person or caregiver. Due to the favorable conductance of the body to sound, the speaker may be audible to the person, caregiver, or neurosurgeon. The speaker may be used to indicate that EEG recording has begun, has been paused, has been stopped. It may indicate that telemetry has begun or has completed. It may indicate the occurrence of an event, such as a seizure. It may indicate a warning condition or hardware fault.

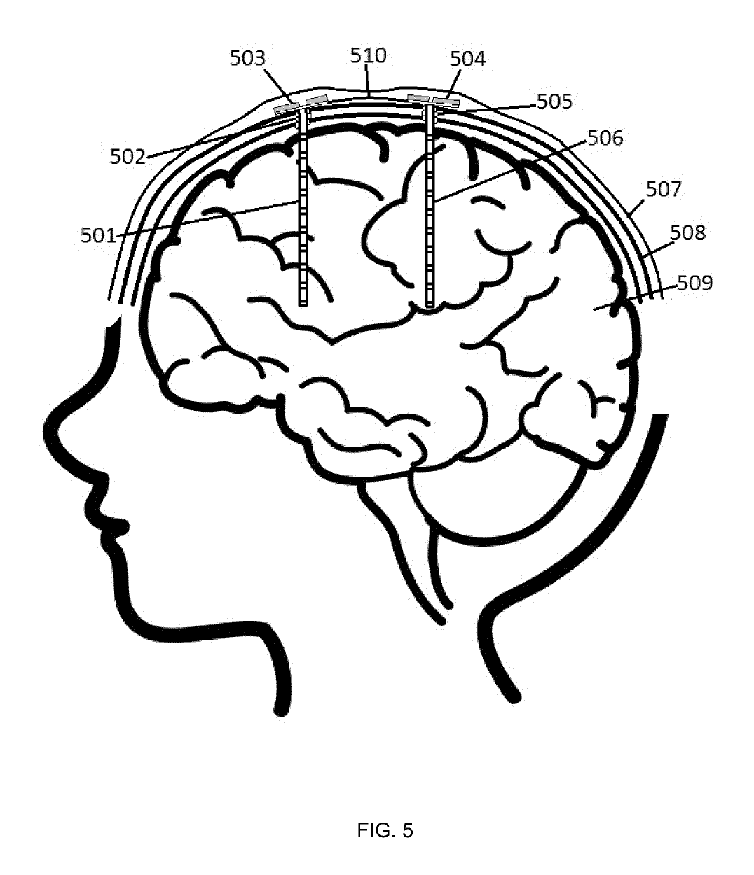

With reference to FIG. 5, a drawing is shown which represents a typical aspect of the present device. In this, two devices are implanted, each with a subcranial probe (501, 506), which penetrates the skull (508) and penetrates the brain (509). Each device has a battery and conductive case (503, 504), which rests against the skull and is underneath the scalp (507). Each device has an intracranial portion that includes a seal (502, 505) to prevent fluid ingress and egress, providing an electrically insulating barrier between the subcranial electrodes and the extracranial electrode. In this configuration, the extracranial electrodes of the two devices are connected together via a conductive wire (510). This allows the extracranial electrode of each device to act as a ground reference, where the potential of the ground reference for each device is identical. This allows an accurate measurement of the potential between electrodes on two different subcranial probes. However, this would require either making an incision to route the wire, or tunneling the wire between the two devices.

In one aspect of the device, the device incorporates a means to administer an electric current pulse between two subcranial electrodes. By administering an electric current pulse, it may be possible to provide functional mapping of the brain and to determine whether a particular region of brain is eloquent, meaning that it is directly involved in motor function, memory, or some other vital function. An electric current pulse could also be used to electrically stimulate a region of cortex, potentially circumventing a seizure. The current pulse could be sinusoidal, half-wave, full-wave, square wave, triangular wave, or another shape.

In one aspect of the device, the battery is rechargeable, where an energy source external to the body is brought in close proximity to the device, and the device comprises recharging circuitry. In this case, the person may recharge the battery at regular intervals, in order to provide continued EEG recording, or the person may recharge the battery whenever an EEG recording is desired. For example, if a person has seizures by an aura, or feeling that a seizure is imminent, the person may use the external energy source to recharge the battery in order to record EEG during the seizure.

In one aspect of the device, the battery is not rechargeable. In this case, the device records an EEG for a period of time. When the device is removed from the person's brain, either the battery may be replaced or the device may be disposed, after the EEG recording has been transferred from the device.

Although the preferred aspect of the device comprises a battery, it may also be possible to use another energy storage module, such as a capacitor, in order to power the device for a period of time. In one aspect of the device, a capacitor is used to store energy, in order to power the device. Using a capacitor may make the device smaller and less expensive. However, a capacitor may not be able to power the device for as long as a battery.

In one aspect of the device, the EEG recording is performed for a period of time. For example, to perform EEG mapping for a person who may be undergoing epilepsy surgery to resect a portion of the brain, several devices may be implanted. After implantation, the person may go home and resume daily activities. The EEG recording is performed continuously during this time. When the person has a seizure, the EEG recording will capture the seizure activity. The person may use a magnet or other means to indicate to the device that it should record a marker with the EEG to indicate the time when the seizure occurred. The person may return to the clinic. If the device incorporates wireless telemetry, the clinic staff may be able to transfer the EEG data from the device to an external module, such as a computer, using an external antenna, such as a small coil or dipole antenna that is positioned above the scalp over the device.

If multiple devices are implanted, then it may be necessary to distinguish one device from another using a telemetry protocol. In such a protocol, a device may have a unique identifier, and the protocol will have a link-initiation in which the device identifier is communicated, and thereafter all communication will be only between the device with the unique identifier and the external communications module. Once the link is broken, a new one may be established with a different device with a different unique identifier.

If the device does not incorporate wireless telemetry, it may also be possible to communicate with the device while implanted using a conductive means, whereby an external module uses electrodes place on the skin to generate electric pulses that can be sensed by the device's electrodes. The device's software could be such that it can differentiate these external pulses from normal EEG signals. The device could then communicate with the external module by providing small electrical pulses through its electrodes, which can be sensed by the external device through its surface electrodes. It is anticipated that such a communications mechanism will have a slower data rate than wireless telemetry, but it may allow the device to be smaller and use less power, since it would not need to incorporate the hardware necessary to communicate wirelessly.

If multiple devices are implanted, then since the locations of the subcranial probes of all devices are known, it may be possible to determine the approximate location of the seizure focus, or start of seizure activity. When resection surgery is considered, it is critical that the portion of the brain removed is not essential for the person to perform necessary activities. For example, if the portion of the brain is responsible for speech, movement, or memory, then resection surgery may not be an option. In order to test for that, it may be necessary to provide electrical stimulation to the area to see what effect it may have on the person. For example, the region may be electrically stimulated while the person speaks, and if speech is inhibited while stimulation is being generated, then that region may not be a candidate for resection. In one aspect of the device, the device incorporates a means to deliver electric pulses between two of the electrodes, in order to test the region's functionality.

It has been shown that stimulation of a region of the brain at the seizure focus when a seizure is imminent may circumvent the seizure. In one aspect of the device, the EEG recording is analyzed by software in the processor in the device, and when it is determined that a seizure is imminent, the device may provide electrical pulses between two electrodes to stimulate the desired region of the brain, thereby circumventing the seizure.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A device, which is fully implantable in a person, and records the EEG of a person, and comprises an energy storage module, control logic, memory, an EEG amplifier, a subcranial electrode, an extracranial electrode, and an intercranial portion, the intercranial portion passing through an opening in the skull and comprising a seal, wherein the seal prevents electric current from passing around the intercranial portion through the opening in the skull.

2. A device of claim 1, where EEG is recorded between two subcranial electrodes, with the extracranial electrode acting as a ground reference.

3. A device of claim 1, where EEG is recorded between the subcranial electrode and the extracranial electrode.

4. A device of claim 1, where the energy storage module is a battery.

5. A device of claim 1, where the energy storage module is a capacitor.

6. A device of claim 1, where the control logic comprises a means whereby the person can record a marker on the EEG recording.

7. A device of claim 6, where the means of recording an EEG marker is initiated by placing a permanent magnet close to the device, which activates a magnetic switch in the device.

8. A device of claim 1, where the control logic comprises a means of sending the recorded EEG data to an external device.

9. A device of claim 8, where the means of sending the recorded EEG is through wireless telemetry.

10. A device of claim 8, where the means of sending the recorded EEG is through a wired connection.

11. The device of claim 1, where the extracranial electrode is electrically connected to the extracranial electrode of a nearby device.

12. The device of claim 1, where the device comprises a speaker to communicate events to a person.

13. The device of claim 12, where the speaker is used to indicate recording start, recording stop, battery level, or EEG marker set.

14. The device of claim 1, where the device comprises a removable anchor shaft that allows for stereotactic implantation of the device.

15. The device of claim 1, where the energy storage module, control logic, memory, EEG amplifier, and extracranial electrode are contained in an enclosure that is located in the extracranial region under the scalp.

16. The device of claim 15, where the container is configured to tilt, to allow the intercranial portion to pass through the skull at an angle that is not perpendicular to the skull.

17. The device of claim 1, where the subcranial electrode is part of a probe, which proceeds a distance into the brain.

18. The device of claim 17, where the probe does not penetrate the dura.

19. The device of claim 17, where the probe penetrates the dura.

20. The device of claim 1, where the device is used to localize seizure focus.
